(19) United States
(12) Patent Application Publication
(10) Pub. No.: US 2008/0004235 A1
(43) Pub. Date: Jan. 3, 2008

(54) TOPICAL COMPOSITIONS COMPRISING ONE OR MORE OF 4-THIOURIDINE, ISOMALTITOL AND URIDINE

(76) Inventor: Srinivas Uppugunduri, Linkoping (SE)

Correspondence Address: GAUTHIER & CONNORS, LLP, 225 FRANKLIN STREET, SUITE 2300, BOSTON, MA, 02110, US

(21) Appl. No.: 11/768,438

(22) Filed: Jun. 26, 2007

Related U.S. Application Data
(63) Continuation of application No. PCT/SE2006/000008, filed on Jan. 2, 2006.

(30) Foreign Application Priority Data
Jan. 3, 2005 (SE) 0500020-3

(51) Int. Cl. A61K 31/7072; A61K 31/7016; A61P 17/04; A61P 17/06; A61P 17/08; A61P 17/10; A61P 17/16; A61P 17/18

(52) U.S. Cl. 514/50; 514/53

(57) ABSTRACT

The present invention relates to the use of one or more of the compounds of the group consisting of 4-thiouridine, isomaltitol, and uridine in the preparation of topical, therapeutically effective compositions for treating psoriasis, atopic and contact dermatitis, eczema, seborrhioea, stasis dermatitis and stasis ulcers, as well as allergic rhinitis, as well as a method for treatment of psoriasis, atopic and contact dermatitis, eczema, seborrhoea, stasis dermatitis and stasis ulcers, as well as allergic rhinitis, with the exception of the use of uridine in the treatment of inflammatory conditions caused by bacterial infections.

### TECHNICAL FIELD

The present invention relates to novel, specific inhibitors of acute and chronic inflammation, method for treatment of acute and chronic inflammation and/or problems in hemostasis related to platelet function.

The object of the present invention is to obtain novel, specific inhibitors of acute and chronic inflammations in order to be able to treat such inflammatory conditions.

### BACKGROUND OF THE INVENTION

Psoriasis is one of the most common dermatologic diseases, affecting up to 1 to 2 percent of the world's population. It is a chronic inflammatory skin disorder clinically characterized by erythematous, sharply demarcated papules and rounded plaques, covered by silvery micaceous scale. The skin lesions of psoriasis are variably pruritic. The most common variety of psoriasis is called plaque type. Patients with plaque-type psoriasis will have stable, slowly growing plaques, which remain basically unchanged for long periods of time. The most common areas for plaque psoriasis to occur are the elbows, knees, gluteal cleft, and the scalp. Involvement tends to be symmetrical. Inverse psoriasis affects the intertriginous regions including the axilla, groin, submammary region, and navel, and it also tends to affect the scalp, palms, and soles. The individual lesions are sharply demarcated plaques but may be moist due to their location. Plaque-type psoriasis generally develops slowly and runs an indolent course. It rarely spontaneously remits.

Eruptive psoriasis (guttate psoriasis) is most common in children and young adults. It develops acutely in individuals without psoriasis or in those with chronic plaque psoriasis. Patients present with many small erythematous, scaling papules, frequently after upper respiratory tract infection with beta-hemolytic streptococci.

The etiology of psoriasis is still poorly understood, but there is clearly a genetic component. Over 50 percent of patients with psoriasis report a positive family history. Evidence has accumulated clearly indicating a role for T cells and neutrophils in the pathogenesis of psoriasis. Psoriatic lesions are characterized by infiltration of skin with activated memory T cells, with CD 8+ cells predominating in the dermis. E-selectin mediated interactions with cutaneous lymphocyte-associated antigen (CLA) are crucial for T-cell homing to the skin. (Schön 2002). Psoriasis may become particularly severe in individuals who are HIV-infected. Stimulation of immune function with cytokines such as interleukin 2 has been associated with abrupt worsening of preexisting psoriasis, and bone marrow transplantation has resulted in clearance of disease. In addition, agents that inhibit activated T cell function are often effective for the treatment of severe psoriasis.

Current therapy of psoriasis depends on the type, location, and extent of disease. All patients should be instructed to avoid excess drying or irritation of their skin and to maintain adequate cutaneous hydration. Most patients with localized plaque-type psoriasis can be managed with midpotency topical glucocorticoids, although their long-term use is often accompanied by loss of effectiveness (tachyphylaxis). Ultraviolet light is an effective therapy for patients with widespread psoriasis. The combination of the UV-A spectrum and either oral or topical psoralens (PUVA) is also extremely effective for the treatment of psoriasis, but long-term use may be associated with an increased incidence of squamous cell cancer and melanoma of the skin. Methotrexate and the synthetic retinoid, acetretin, have been used in patients with severe psoriasis, but toxic side-effects limit their long-term use.

For many patients psoriasis remains a severe, crippling and potentially life threatening disease.

Eczema, or dermatitis, is a reaction pattern that presents with variable clinical and histological findings; it is the final common expression for a number of disorders including atopic dermatitis, allergic contact and irritant contact dermatitis, dyshidrotic eczema, nummular eczema, lichen simplex chronicus, asteatotic eczema, and seborrheic dermatitis.

Atopic dermatitis a common skin disease, especially in children, is associated with a positive family history of asthma, hay fever, or dermatitis in up to 70 percent of patients. It has been estimated that approximately 10 percent of all children will manifest some form of atopic eczema. Atopic dermatitis may resolve spontaneously in adults, but the dermatitis will persist into adult life in over half of individuals affected as children.

Although there is a clear genetic component, the etiology of AD is only partially defined. AD is a chronic allergic inflammatory disease characterized by chronic relapsing inflammation and symmetrical skin lesions. Epidemiological studies indicate the onset of AD is influenced by environmental factors and that its prevalence is increasing (Aioi et al 2001). Dry skin and impairment of skin barrier function have been reported as dermatological features of AD. Histological examination of the skin affected by AD shows similarities to skin affected by acute or chronic dermatitis. Immunopathology shows activated memory T helper cells, which express cutaneous lymphocyte antigen (CLA), the ligand for E-selectin induced on endothelial cells. Elevated levels of IgE against inhaled allergens, elevated levels of CD4+ T cells generating IL-4 are also involved in the pathogenesis of AD (Sengoku 1999, Aioi 2001).

Therapy of atopic dermatitis should be based on avoidance of cutaneous irritants, adequate cutaneous hydration, judicious use of low- or midpotency topical glucocorticoids, and prompt treatment of secondarily infected skin lesions. Antihistamines are useful to control the pruritus that accompanies eczema, but sedation may limit their usefulness. Treatment with systemic glucocorticoids should be limited to severe exacerbations unresponsive to conservative topical therapy. In the patient with chronic atopic eczema, therapy with systemic glucocorticoids will generally clear the skin only briefly, but cessation of the systemic therapy will invariably be accompanied by return, if not worsening, of the dermatitis.

Contact dermatitis is an inflammatory process in skin caused by an exogenous agent or agents that directly or indirectly injure the skin. This injury may be caused by an inherent characteristic of a compound [irritant contact dermatitis (ICD)]. Agents that cause allergic contact dermatitis (ACD) induce an antigen-specific immune response. The prevalence of contact dermatitis in western industrialized nations may be as high as 5 to 20 percent, with ICD being much more common than ACD. The most common presentation of contact dermatitis is hand eczema, and it is frequently related to occupational exposures. ACD is a manifestation of delayed type hypersensitivity mediated by memory T lymphocytes in the skin.

Treatment of ACD and ICD should be directed to avoidance of irritants and the use of protective clothing. Usually, treatment with a high-potency fluorinated topical glucocorticoid is enough to relieve symptoms. Patients with particularly widespread disease, or disease involving the face or genitalia, may require treatment with oral glucocorticoids.

Hand eczema is a very common, chronic skin disorder. It represents the overwhelming majority of occupation-associated skin disease, which is responsible for a significant proportion of occupation-associated injury and time lost from work.

Therapy of hand dermatitis is directed toward avoidance of irritants, identification of possible contact allergens, treatment of coexistent infection, and application of mid- to high-potency topical glucocorticoids.

Stasis dermatitis develops on the lower extremities secondary to venous incompetence and chronic edema. Early findings in stasis dermatitis may consist of mild erythema and scaling associated with pruritus. The typical initial site of involvement is over the medial aspect of the ankle, often over a distended vein. Stasis dermatitis is often complicated by secondary infection and contact dermatitis. Severe stasis dermatitis may precede the development of stasis ulcers.

Avoidance of irritants and use of emollients and/or midpotency topical glucocorticoids are the cornerstones of therapy for stasis dermatitis. Stasis ulcers are difficult to treat, and resolution of these lesions is slow even under the best of circumstances. Glucocorticoids should not be applied to ulcers, since they may retard healing. Some ulcers may take months to heal or require skin grafting.

Allergic rhinitis is characterized by sneezing; rhinorrhea; obstruction of the nasal passages; conjunctival, nasal, and pharyngeal itching; and lacrimation, all occurring in a temporal relationship to allergen exposure. The incidence of allergic rhinitis in North America is about 7 percent, with the peak occurring in childhood and adolescence.

Allergic rhinitis generally presents in atopic individuals, i.e., in persons with a family history of a similar or related symptom complex and a personal history of collateral allergy expressed as eczematous dermatitis, urticaria, and/or asthma. Episodic rhinorrhea, sneezing, and obstruction of the nasal passages with lacrimation and pruritus of the conjunctiva, nasal mucosa, and oropharynx are the hallmarks of allergic rhinitis. Biopsy specimens of nasal mucosa during an episodic allergic reaction show profound submucosal edema with infiltration predominantly by eosinophils, although some neutrophil polymorphonuclear leukocytes are present. The nasal secretions of allergic patients are rich in eosinophils, and peripheral eosinophilia with elevations in relation to clinical exacerbations is a common feature. Total serum IgE is frequently elevated, but the demonstration of immunologic specificity for IgE is critical to an etiologic diagnosis.

Management with pharmacological agents represents the standard approach to seasonal or perennial allergic rhinitis. Antihistamines of the H₁ class are effective for nasopharyngeal itching, sneezing, and watery rhinorrhea and for such ocular manifestations as itching, tearing, and erythema, but they are not efficacious for the nasal congestion, The older antihistamines are sedating, and their anticholinergic (muscarinic) effects include visual disturbance, urinary retention, and even arrhythmias. Alpha-adrenergic agents such as phenylephrine or oximetazoline are generally used topically to alleviate nasal congestion and obstruction, but the duration of efficacy is limited because of rebound rhinitis and such systemic responses as insomnia, irritability, and hypertension.

Intranasal high-potency glucocorticoids are the most potent drugs available for the relief of established rhinitis, seasonal or perennial, and even vasomotor rhinitis; they provide efficacy with substantially reduced side effects as compared with this same class of agent administered orally. Their most frequent side effect is local irritation, with Candida overgrowth being a rare occurrence. Topical high-potency glucocorticoids exhibit superior efficacy as compared to antihistamines throughout the pollen seasons.

Three families of cell-adhesion molecules (CAMs) have been implicated in mediating interactions of platelets, endothelial cells, and leukocytes: the selectins, the integrins and the immunoglobulin superfamily. The selectin family of molecules comprising E-, L-, and P-selectin act in concert with other cell adhesion molecules to effect adhesive interactions of platelets, endothelial cells, and leukocytes. Extensive literature is available implicating cell adhesion molecules in diverse disease processes including reperfusion injury, cancer, coronary heart disease, atherosclerosis, and restenosis after coronary angioplasty, and chronic inflammatory diseases like asthma and IBD.

The emigration of white blood cells to inflammatory sites requires at least four steps: leukocyte rolling along activated endothelium, leukocyte activation, firm adhesion and transendothelial migration. Interaction of the selectins with their carbohydrate ligands seems to be important for the initial binding of the leukocytes to the endothelium under conditions of fluid shear stress. Subsequent firm adhesion and extravasation seems to be mediated by another family of molecules, the β2 (CD 18)-integrins. A number of soluble mediators like IL-1β, TNF, endotoxin, thrombin and histamine can up regulate one or more endothelial adhesion molecules. The major adhesion receptors and ligands regulating leukocyte-endothelium interactions include ICAM-1, ICAM-2/LFA-1; VCAM/VLA-4; L-selectin/GlyCAM-1; CD 34, MAdCAM-1; E-selectin/ESL-1, PSGL-1; P-selectin/PSGL1.

Genetic experiments involving construction and testing of mice deficient in each of these selectins suggest that they provide overlapping, but crucial contributions to leukocyte recruitment in inflammation. Mice lacking each of these selectins exhibit defects in neutrophil rolling and extravasation. Further, mice deficient in both E and P selectin present extreme leukocytosis, elevated cytokine levels and alterations in haematopoesis.

Platelet P-selectin may also play a very important role in both hemostasis and the ensuing inflammatory reaction. Platelets are rapidly recruited to the site of the vascular injury to prevent excessive bleeding. The interaction of platelets with the vessel wall is a crucial event leading to the formation of a hemostatic plug. The role of platelet P-selectin in hemostasis was confirmed in a recent study which showed that platelets roll on stimulated endothelium expressing P-selectin in vivo. Furthermore, bleeding time was prolonged by 40% in P-selectin deficient mice.

Today the treatment of inflammatory conditions include treatment with steroids which negatively affect the immune system and at times leads to a total inhibition of the immune system. It is therefore of interest to control and modulate the inflammatory response.

WO 96/01115 relates to pyrimidine nucleotide precursors for treatment of systemic inflammation and encompasses uridine. The systemic inflammations are caused by bacterial sepsis.

Inflammation is a common feature in the pathogenesis of numerous diseases. Inflammation is normally localized defensive response to invasion of the host by foreign material. Inflammation can be caused by a wide variety of agents including mechanical trauma, toxins, and neoplasia and is not a response reserved exclusively for bacterial infections. The accumulation and subsequent activation of leukocytes are, however, central events in the pathogenesis of virtually all forms of inflammation.

The knowledge of a protective effect of uridine in bacterial sepsis does not automatically lead to the deduction that uridine could benefit in inflammation caused by other agents. Similarly, although it is known that bacterial sepsis can lead to coagulation problems, a number of bleeding disorders are associated with platelet dysfunction without a concomitant bacterial infection. Since bacteria are not involved in the etiology of a number of diseases covered by the present use including reperfusion injury, cancer, coronary heart disease, arteriosclerosis, restenosis after coronary angioplasty, and chronic inflammatory diseases like asthma, rheumatoidal diseases like rheumatoid arthritis, and IBD.

Rational Behind Random In Vitro Screening

Since initial binding of leukocytes to endothelium initiates the inflammatory process, it has been utilized in a vitro system to study the adhesion of various cells to human umbilical cord endothelial cells. The method is well established and has been successfully utilized by a number of research groups. This in vitro static adhesion assay has been used herein for random screening of substances that can block leukocyte adhesion to endothelial cells. A variety of substances were tested as literature has shown that a number of, both, carbohydrate and non-carbohydrate structures can block interaction of selectins with cognate ligands. Substances that can block interaction of endothelial adhesion molecules with cognate ligands on leukocytes have potential as novel, selective inhibitors of acute and chronic inflammatory reactions and ischemia reperfusion states, without risk of general immuno-suppression.

### DESCRIPTION OF THE PRESENT INVENTION

Screening of various chemical entities resulted in identification of three lead compounds that could be used in the preparation of pharmaceutical compositions for topical administration for treating all and varied symptoms of psoriasis, atopic and contact dermatitis, eczema, seborrhoea, stasis dermatitis and stasis ulcers, as well as allergic rhinitis, symptoms of irritation, dryness, itching, erythema and swelling due to occupational exposure to irritants or as a result of exposure to UV light including sunshine and artificial lamps, or as a result of various cosmetological procedures including so called peeling. Eczema, or dermatitis is the final common expression for a number of disorders including atopic dermatitis, allergic contact and irritant contact dermatitis, dyshidrotic eczema, nummular eczema, lichen simplex chronicus, asteatotic eczema, and seborrheic dermatitis. Hand eczema can also be related to occupational exposure to various irritants. The compounds obtained in this screening were: isomaltitol, uridine and 4-thiouridine.

The invention will be described more in detail in the following with reference to screening tests performed. The test results are also illustrated in the accompanying graphs.

The compound isomaltitol is often present in a mixture of called isomalt, which is a sugar substitute consisting of disaccharide alcohols. It is derived exclusively from sucrose by enzymatic conversion into isomaltulose, which is then hydrogenated to obtain a 1:1 mixture of two disaccharide alditols (disaccharide sugar alcohol's)—glucomannitol (6-O-α-D-glucopyranosyl-D-mannitol, GPM) and isomaltitol (6-O-α-D-glucopyranosyl-D-sorbitol, glucosorbitol, GPS,) (Cataldi, Campa et al. 1999) (FIG. 1). One of these compounds, the isomaltitol, (GPS) was used in all our experimental work and was obtained from Sigma Corp., St. Louis, USA.

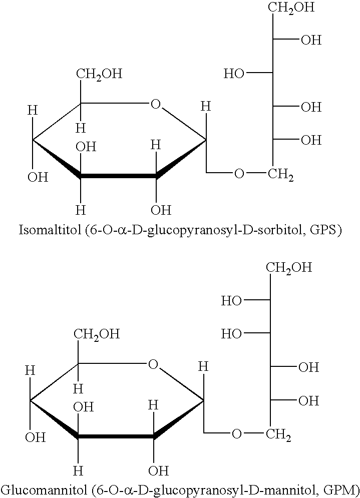

Isomalt is available as a crystallized powder. As a consequence of the conversion of the reducing sugar group of the intermediate isomaltulose into an alcohol function during the production of isomalt, the chemical stability is improved and the tendency to undergo Maillard reactions (browning) is substantially reduced. This process also modifies several physicochemical properties such as solubility, viscosity, hygroscopicity and boiling temperature, parameters. Isomalt has a molecular weight of 344.

Isomaltitol was discovered in the 1960s, it has been used in Europe as a sweetener in confectionery products since the early 1980s. Isomaltitol doesn't promote any dental caries because almost all bacteria in the mouth are incapable of converting the sugar replacer into decay causing acids (Siebert, Grupp et al. 1975). Isomaltitol causes only a very small rise in blood glucose and insulin levels compared to sugars and other carbohydrates, and this because its absorption is very slow and limited. The disaccharide alcohol doesn't seem to have any toxic effect when tested on rats (Siebert, Grupp et al. 1975).

It is further contemplated that other major inflammatory conditions can be treated using a topical composition of the invention as well. Such conditions are asthmatic conditions, Crohn's disease, ulcerous colitis, reperfusion injury, auto-immune diseases, inflammatory bowl disease (IBD), arteriosclerosis, restenosis, skin cancer, coronary heart disease, diabetes, rheumatoidal diseases, inflammation related to the eye, ear or nasal orifices, dermatological diseases, such as psoriasis, seborrhoea, burn injury, graft rejection. The term topical composition is deemed to encompass treatment of other orifices using eye drops, ear drops or rectal administration using for example a rectal clysma or suppository,

Animal Models Used for Proof of Principle Test

Animal Models for Psoriasis:

T Cell-Mediated Mouse Psoriasis Model

Human-to-SCID Mice Xenotransplantation Model

Human psoriatic plaques are engrafted to severe combined immunodeficient mice followed by injection of activated immunocytes.

Spontaneous Mutation Mice Models

There are several spontaneous mouse mutations with psoriasiform skin alterations of unclear pathogenesis, for example asebia (ab), flaky skin (fsn) and chronic proliferative dermatitis (cpd).

Animal Models for Dermatitis and Eczema

NC/Nga Tnd Mice as a Model for Atopic Dermatitis

One of several unusual biological characteristics of the NC/Nga Tnd mice is the appearance of spontaneous dermatitis when the mice are raised under ambient laboratory conditions (ALC), i.e. raised in laboratories without filtration of the circulating air.

Tuberculin-Induced Delayed Allergic Reaction in Actively Sensitized Mice

Ovalbumin-Sensitized/IgE Rat Model

Mite Antigen-Sensitized Mouse Model

Mice are sensitized with mite antigen by a single topical application to barrier-disrupted abdominal skin.

Mite Antigen-Sensitized Mouse Ear Model

The ears of SPF NC/Nga mice are intradermally injected with extracts of mite antigen. This results in atopic dermatitis-like skin lesions.

IL-4 Transgenic Mice-Model

(The transgenic mice spontaneously develop a pruritic inflammatory skin disease reproducing all key features of human atopic dermatitis.)

Methodological Study on Humans

Skin irritation and dermatitis is a common problem among health care professionals due to frequent washing. We have utilized two well-established skin irritation models to investigate the effect of topically administered uridine or isomalt on skin physiology including measurement of dermal blood flow. Isomalt consists of 1,6-glucopyranosyl-D-sorbitol (GPS, isomaltitol) and 1,1-glucopyranosyl-D-mannitol (GPM) in a 1:1 ratio. The methodological study involves the study of various physiological parameters, such as dermal blood flow and visual assessment of erythema, subsequent to provocation by either exposure to UVB light or Sodium Lauryl Sulphate (SLS) and the effects of isomalt and uridine on these parameters. Only a very small area of the skin was exposed to the irritants UVB or SLS. The methodological study has been approved by the Medical Products Agency, Sweden and the Regional Ethical Review Board, Linköping, Sweden.

Ultraviolet B Irradiation Induced Skin Erythema and SLS Standard Irritant Model

UVB provocation with a divergent beam produces a circular, radially fading skin erythema, the diameter of which corresponds to the minimal erythemal dose. The type of skin damage is comparable to that seen after excessive sunbathing. SLS, which is a detergent, creates a local skin irritation mainly by disturbing the skins barrier function. A methodological study has been performed, both to evaluate the effects of uridine or isomalt on skin UVB-induced erythema and SLS irritation. The primary objective of the study was to make a preliminary safety evaluation.

The following test areas were studied on each subject:
Area 1: Provocation with SLS or UVB light, respectively
Area 2: Provocation with SLS or UVB light, respectively, and treatment with vehicle
Area 3: Provocation with SLS or UVB light, respectively, and treatment with a saturated solution of uridine
Area 4: Provocation with SLS or UVB light, respectively, and treatment with a saturated solution of isomalt

Dermatitis, or even a mild skin irritation, leads to an increased blood flow through the skin that can only sometimes be registered by the human eye. The effect of various treatments was registered by measurement of dermal blood flow in the erythema, both visually and by Laser Doppler Imaging (LDPI). LDPI is a non-invasive technique for measurement of blood flow that does not interfere with the microcirculation and is more sensitive and objective than a visual assessment (Fullerton et al. 2002, Serup 1995). The visual assessment was performed according to York et al (1996) as follows:

0 No reaction

+ Weak reaction

++ Mild reaction

+++ Strong reaction

Results:

All individuals exposed to a saturated solution of uridine or isomalt for 24 hours showed no untoward effects, such as exacerbated skin irritation or itching related to the exposure. In all, 7 of the 8 healthy volunteers exposed to UVB irradiation, had a smaller area of erythema when the irradiated skin area was exposed to isomalt compared to those where the vehicle was used. Isomalt induced a decrease in erythemal diameter in 7 out of 8 healthy volunteers with a mean decrease of 19.7 % of afflicted area. We are currently conducting an analysis of mean perfusion rates for an objective comparison of treatments and mathematical assessment of changes in minimal erythemal dose and UVB dose response.

By “pharmaceutically acceptable,” such as in the recitation of a “pharmaceutically acceptable carrier,” or a “pharmaceutically acceptable derivative,” is meant a compound that is not biologically or otherwise undesirable, i.e., the compound may be incorporated into a topical formulation of the invention and administered to a patient without causing any undesirable biological effects or interacting in a deleterious manner with any of the other components of the formulation in which it is contained. A “pharmacologically active” compound refers to an active agent as defined above, or to an analog or derivative thereof having the same type of pharmacological activity as the parent compound.

The terms “treating” and “treatment” as used herein refer to actions that reduce the severity and/or frequency of symptoms, eliminate symptoms and/or their underlying cause, prevent the occurrence of symptoms and/or their underlying cause, and improve or remediate damage. The present method of “treating” a patient, as the term is used herein, thus encompasses both prevention of indicated symptoms and signs in a predisposed individual and treatment of the indicated symptoms and signs in a clinically symptomatic individual.

By an “effective” amount or a “therapeutically effective amount” of a pharmacologically active agent is meant a non-toxic but sufficient amount of the drug or agent to provide the desired effect, i.e., prevention or treatment of the symptoms and signs given in the present claims. The amount that is “effective” will vary from subject to subject, depending on the age and general condition of the individual, mode of administration, and the like. Thus, it is not always possible to specify an exact “effective amount.” However, an appropriate “effective” amount in any individual case may be determined by one of ordinary skill in the art using routine experimentation.

The term “topical administration” is used in its conventional sense to mean delivery of a topical drug or pharmacologically active agent to the skin or mucosal tissue, as in, for example, the treatment of the symptoms and signs given in the present claims.

The term “body surface” is used to refer to skin or mucosal tissue.

“Carriers” or “vehicles” as used herein refer to pharmaceutically acceptable carrier materials suitable for topical drug administration. Carriers and vehicles useful herein include any such materials known in the art that are nontoxic and do not interact with other components of the composition in a deleterious manner.

The term “aqueous” refers to a formulation that contains water or that becomes water-containing following application to the skin or mucosal tissue.

In describing molecular structures and formulae herein, the phrase “having the formula” or “having the structure” is not intended to be limiting and is used in the same way that the term “comprising” is commonly used.

Further, as used herein, the term “safe and effective amount” refers to the quantity of a component, which is sufficient to yield a desired therapeutic response without undue adverse side effects commensurate with a reasonable benefit/risk ratio when used in the manner of this invention.

“Adverse side effects” include, without limitation, toxicity, irritation, and allergic response. The specific “safe and effective amount” will, therefore, vary with such factors as the particular condition being treated, the physical condition of the patient, the duration of the treatment, the nature of concurrent therapy, if any, and the specific formulations employed.

As used herein, the term “daily dosage” refers to an amount of material administered to a patient during each day of the treatment. When the material is administered topically, daily dosages are assumed to apply to a treatment area measuring 100 cm², unless another size of treatment area is specified.

In certain embodiments of the invention, methods for the reduction, treatment, or at least partial prevention of eczema and/or psoriasis are provided. In the methods of the present invention, a composition comprising a therapeutically effective amount of an active compound is administered to a patient afflicted with eczema and/or psoriasis. The composition may optionally include a pharmaceutically acceptable carrier. The composition may also optionally include any antioxidant. Preferably, the/composition used in the present invention is administered topically.

It has now surprisingly been found that the methods of the present invention are effective to reduce, treat or at least partially prevent eczema and/or psoriasis. The compositions used in the methods of the present invention include at least one active compound.

In a method for treating or reducing eczema and psoriasis, an effective amount of the topical composition comprising an active compound is applied one to twelve times daily, as needed, to an area of skin afflicted with eczema and/or psoriasis. Preferably, the composition is applied one to six times daily, and, more preferably, two or three times daily. In the method, a thin layer of the topical composition is applied to the inflicted area of skin, by rubbing, spraying, pouring, spreading, or a like method. Also preferably, the topical composition is rubbed into the skin until little or no residue remains on the skin.

The pharmaceutically acceptable carrier used in the present invention may be a carrier suitable for use as a carrier for topical compositions. The non-carrier ingredients are dissolved, dispersed and/or suspended in the topical composition. Examples of suitable topical carriers include, without limitation, creams, ointments, lotions, pastes, jellies, sprays, aerosols, bath oils, and other topical pharmaceutical carriers, which accomplish direct contact between the active ingredients of the topical composition of the present invention and the pore of the skin. Preferably, the pharmaceutically acceptable topical carrier may make up more than about 80%, and more preferably about 80-95% w/w of the total composition. In some cases, it may be necessary to dissolve one or more the active ingredients in an appropriate solvent such as ethanol or DMSO (dimethyl sulfoxide), or the like, to facilitate the incorporation of one or more active ingredients into the topical composition or the pharmaceutically acceptable topical carrier.

Preferably, an amount of topical carrier sufficient to provide a substantially homogeneous cream or ointment is used.

One preferred topical carrier useful in the present invention contains at least a hydrophilic ointment base, panthenol or a panthenol derivative and a dispersant if needed to disperse one or more insoluble or partially insoluble active ingredients in the carrier.

Another preferred topical carrier of the present invention employs hydroxymethyl cellulose as the base and may contain ingredients contained in the carrier described below other than the hydrophilic ointment base.

Yet another preferred pharmaceutically acceptable topical carrier includes a solution of an acrylic copolymer in a non-aqueous solvent system, which mainly contains polyethylene glycol such as methoxy polyethylene glycol 550 (MPEG). A particular preferred MPEG is Sentry Carbowax MPEG 550 sold by the Dow Chemical Company of Midland, Mich., which is a food/pharmaceutical/cosmetic grade material. Polyethylene glycols are generally non-toxic, water-soluble polymers that are fully biodegradable. In the solution, the acrylic copolymer would preferably be present in a concentration range of 3-6% by weight. Preferably, the acrylic copolymer has a molecular weight of more than 20,000 Daltons. More preferably, the acrylic copolymer has a molecular weight of more than 100,000 Daltons. Acrylic polymers having higher molecular weights are less likely to be absorbed by the human body or skin. Other pharmaceutically acceptable carrier components, other than the hydrophilic ointment base may also be employed in this carrier material, if compatible with the acrylic copolymer.

Suitable hydrophilic ointment bases are known to persons skilled in the art.

Examples of hydrophilic ointment bases suitable for use in the present invention include, without limitation, non-U. S. P. hydrophilic ointment bases such as those made by Fougera, Inc., of Melville, N.Y. Sufficient hydrophilic ointment base is employed to act as a topical carrier for the active or non-carrier ingredients of the topical composition.

Panthenol or panthenol derivatives are preferably included in the pharmaceutically acceptable carrier. The panthenols useful in the present invention include at least D-panthenol, DL-panthenol and mixtures thereof. This component of the topical carrier has skin moisturizing properties and acts as a quick, deep penetrating component of the topical carrier that helps deliver the non-carrier ingredients through the skin to the area to be treated and may also impart a healing effect to damaged tissue. The amount of panthenol or panthenol derivative to be employed is from about 0.25 to about 10 weight percent, more preferably from about 0.5 to about 5 weight percent and most preferably from about 1 to about 2 weight percent, based on the total weight of the topical composition.

The topical carrier of the present invention may also include additional ingredients such as other carriers, moisturizers, humectants, emollients, dispersants, radiation blocking compounds, particularly UV-blockers, as well as other suitable materials that do not have a significant adverse effect on the activity of the topical composition. Preferred additional ingredients for inclusion in the topical carrier are sodium acid phosphate moisturizer, witch hazel extract, glycerine humectant, other humectants such as Ajidew NL-50, available from Ajinomoto USA, Inc., of Paramus, N.J., apricot kernel oil emollient, and corn oil dispersant.

The topical composition used in the present invention may also be employed to facilitate wound healing. The compositions may also be used to cleanse, beautify, and improve the cosmetic appearance of the skin. In fact, one measure of a therapeutically effective amount of the various compositions of the present invention is by determining if that amount improves the healing of, or the cosmetic appearance of the skin. More preferably, the compositions of the present invention are employed to improve the healing of, or the cosmetic appearance of the skin in the area afflicted by reactive and inflammatory dermatoses such as eczema and/or psoriasis.

More preferably, an effective amount of the topical composition comprising an active compound is applied to an area of skin afflicted with reactive and inflammatory dermatoses such as eczema and/or psoriasis. Preferably, the composition is applied one to six times daily, and, more preferably, two or three times daily. In the method, a thin layer of the topical composition is applied to the inflicted area of skin, by rubbing, spraying, pouring, spreading, or a like method. Also preferably, the topical composition is rubbed into the skin until little or no residue remains on the skin.

In a method for cleansing, beautifying, and improving the cosmetic appearance of the skin, an effective amount of the topical composition comprising am active compound is applied one to twelve times daily, as needed, to an area of skin. More preferably, an effective amount of the topical composition comprising an active compound is applied to an area of skin afflicted with reactive and inflammatory dermatoses such as eczema and/or psoriasis.

The method can also be applied to alleviate symptoms of irritation, dryness, itching, erythema and swelling due to occupational exposure to irritants or as a result of exposure to UV light including sunshine and artificial lamps, or as a result of various cosmetological procedures including so called peeling.

Preferably, the composition is applied one to six times daily, and, more preferably, two or three times daily. In the method, a thin layer of the topical composition is applied to the inflicted area of skin, by rubbing, spraying, pouring, spreading, or a like method. Also preferably, the topical composition is rubbed into the skin until little or no residue remains on the skin.

The topical composition of the present invention is preferably made by cold compounding. This is an important feature of the invention when one or more of the compounds employed in the topical composition is sensitive to heat or other types of energy. In these cases the activity of the topical composition may be detrimentally affected as a result of the formulation of the topical compositions in a manner that allows excess heat to be generated during compounding. It may be necessary to dissolve, disperse or suspend one or more of the ingredients prior to cold compounding in order to ensure substantially homogeneous distribution of the non-carrier or active ingredients in the topical composition.

A preferred pharmaceutically acceptable topical carrier of the invention can be made using the following ingredients: 25-35 parts of a 50% aqueous solution of sodium acid phosphate moisturizing agent, 5-10 parts of D-or DL-panthenol, 5-10 parts of glycerine, 1-3 parts of apricot kernel oil and 10-20 parts of witch hazel extract.

Optionally, one or more of the optionally ingredients of the topical composition such as glycerin, witch hazel extract, vitamins A and E and/or the ascorbyl palmitate can be reduced or eliminated from a particular topical composition, if desirable, or larger amounts of one type of component, i. e. an antioxidant, can be employed while reducing the amount of another component of the same type or having a similar activity.

When the composition of the present invention is formulated into a topical composition, preferably, the vitamins A and D3 if used in the composition of the present invention may be formulated in a single corn oil dispersion. Generally, every cubic centimeter (cc) of the corn oil dispersion of vitamins A and D3 used in the present invention may contain about 500,000 to about 2,000,000 IU of vitamin A and about 50,000 to about 200,000 IU of vitamin D3. Preferably, every cc of the corn oil dispersion of vitamins A and D3 used in the present invention may contain about 1,000,000 IU of vitamin A and about 100,000 IU of vitamin D3.

Preferably, the active compound is used in an amount of about 2 to about 100 grams per pound of the composition. More preferably, the active compound is employed in an amount of about to about 10-50 grams per pound of the composition, and, still more preferably, about 15 to about 40 grams per pound of the composition. Any antioxidant component in the topical composition is used in an amount of about 100 mg-50 gm, more preferably of about 250 mg-10 gm, and most preferably of about 500 mg-5 gm per one pound of topical base.

Based on 1 lb of hydrophilic ointment base, optional ingredients in the composition may include Adijew™ NL-50 (50% aq., about 10 to about 100 cc, more preferably 25-35 cc), DL panthenol (about 1 to about 50 cc, more preferably about 5 to about 10 cc), glycerin USP (about 1 to about 20 cc, more preferably about 5 to about 10 cc), apricot kernel oil (about 0.1 to about 10 cc, more preferably about 1 to about 3 cc), vitamins A and D3 (as a dispersion in corn oil, about 1×10 IU/g to about 2×10⁷ IU/g of dispersion of vitamin A, more preferably about 6×106 IU/g to about 1×10⁷ IU/g of dispersion of vitamin A; and 1×10⁵ IU/g to about 2×10⁶ IU/g of dispersion of vitamin D3, more preferably about 6×10⁵ IU/g to about 1×10⁵ IU/g of dispersion of vitamin D3, witch hazel extract (about 1 to about 50 cc, more preferably about 10 to about 20 cc), vitamin E acetate (1 g=1000 U, about 0.1 to about 20 cc, more preferably about 1 to about 4 cc), and ascorbyl palmitate (about 0.2 to about 10 g, more preferably, about 2 to about 4 g).

The above-mentioned composition is applied to the afflicted area of the skin in an amount of approximately at least 0.5 ml per 100 cm² per day, preferably at least about 1 ml per 100 cm² per day, and more preferably about 15 ml per 100 cm² per day.

Preferably, the daily dosage does not exceed about 35 ml per 100 cm² per day.

When vitamin A is a component of the composition, it is administered in an amount of at least about 7000 IU/100 cm²/d, preferably at least about 14,000 IU/100 cm²/d, and more preferably about 200,000 IU/100 cm²/d. Preferably, the dosage of vitamin A does not exceed about 500,000 IU/100 cm²/d.

When vitamin D3 is a component of the composition, it is administered in an amount of at least about 500 IU/100 cm2/d, preferably at least about 1000 IU/100 cm2/d, and more preferably about 20,000 IU/100 cm2/d. Preferably, the dosage of vitamin D3 does not exceed about 50,000 IU/100 cm2/d.

When vitamin E is a component of the composition, it is administered in an amount of at least about 2 IU/100 cm2/d, preferably at least about 5 IU/100 cm2/d, and more preferably about 75 IU/100 cm2/d. Preferably, the dosage of vitamin E does not exceed about 150 IU/100 cm2/d.

When ascorbyl palmitate is a component of the composition, it is administered in an amount of at least about 2.5 mg/100 cm2/d, preferably at least about 5 mg/100 cm2/d, and more preferably about 75 mg/100 cm2/d. Preferably, the dosage of ascorbyl palmitate does not exceed about 150 mg/100 cm2/d.

The total amount of active compounds administered in the composition is at least about 3 mg/100 cm²/d, preferably at least about 6 mg/100 cm²/d, and more preferably about 100 mg/100 cm²/d. Preferably, the dosage of all active compounds combined does not exceed about 200 mg/100 cm²/d.

The compositions of the present invention may also be formulated into a spray, mouth rinse, aerosol or inhalant. Such compositions may be prepared using well-known techniques. For these types of formulations, suitable carriers may include the following ingredients: saline with one or more preservatives, absorption promoters to enhance bioavailability, fluorocarbons and/or other conventional solubilizing or dispersion agents.

These compositions are specialized forms of the topical compositions of the present invention and are designed for topical application to eczema or psoriasis including eczema or psoriasis that may occur in the mouth or nasal cavity.

The following examples are provided to describe the invention in further detail.

These examples, which set forth a preferred mode presently contemplated for carrying out the invention, are intended to illustrate and not to limit the invention.

The formulation may be in any form suitable for application to the body surface, and may comprise, for example, a cream, lotion, solution, gel, ointment, paste, plaster, paint, bioadhesive, or the like, and/or may be prepared so as to contain liposomes, micelles, and/or microspheres. For those formulations in which the pharmacologically active base is a base, it is preferred although not essential that water be present. Thus, such a formulation may be aqueous, i.e., contain water, or may be nonaqueous and optionally used in combination with an occlusive overlayer so that moisture evaporating from the body surface is maintained within the formulation upon application to the body surface and thereafter.

Formulations of the invention may optionally contain a pharmaceutically acceptable viscosity enhancer and/or film former. A viscosity enhancer increases the viscosity of the formulation so as to inhibit its spread beyond the site of application. Balsam Fir (Oregon) is an example of a pharmaceutically acceptable viscosity enhancer.

A film former, when it dries, forms a protective film over the site of application. The film inhibits removal of the active ingredient and keeps it in contact with the site being treated. An example of a film former that is suitable for use in this invention is Flexible Collodion, USP. As described in Remington: The Science and Practice of Pharmacy, 19th Ed. (Easton, Pa.: Mack Publishing Co., 1995), at page 1530, collodions are ethyl ether/ethanol solutions containing pyroxylin (a nitrocellulose) that evaporate to leave a film of pyroxylin. A film former may act additionally as a carrier. Solutions that dry to form a film are sometimes referred to as paints.

Ointments, as is well known in the art of pharmaceutical formulation, are semisolid preparations that are typically based on petrolatum or other petroleum derivatives. The specific ointment base to be used, as will be appreciated by those skilled in the art, is one that will provide for optimum drug delivery, and, preferably, will provide for other desired characteristics as well, e.g., emolliency or the like. As with other carriers or vehicles, an ointment base should be inert, stable, nonirritating and nonsensitizing. As explained in Remington: The Science and Practice of Pharmacy, 19th Ed. (Easton, Pa.: Mack Publishing Co., 1995), at pages 1399-1404, ointment bases may be grouped in four classes: oleaginous bases; emulsifiable bases; emulsion bases; and water-soluble bases. Oleaginous ointment bases include, for example, vegetable oils, fats obtained from animals, and semisolid hydrocarbons obtained from petroleum. Emulsifiable ointment bases, also known as absorbent ointment bases, contain little or no water and include, for example, hydroxystearin sulfate, anhydrous lanolin and hydrophilic petrolatum. Emulsion ointment bases are either water-in-oil (W/O) emulsions or oil-in-water (O/W) emulsions, and include, for example, cetyl alcohol, glyceryl monostearate, lanolin, and stearic acid. Preferred water-soluble ointment bases are prepared from polyethylene glycols of varying molecular weight; again, see Remington: The Science and Practice of Pharmacy for further information.

Creams, as also well known in the art, are viscous liquids or semisolid emulsions, either oil-in-water or water-in-oil. Cream bases are water-washable, and contain an oil phase, an emulsifier, and an aqueous phase. The oil phase, also called the “internal” phase, is generally comprised of petrolatum and a fatty alcohol such as cetyl or stearyl alcohol. The aqueous phase usually, although not necessarily, exceeds the oil phase in volume, and generally contains a humectant. The emulsifier in a cream formulation is generally a nonionic, anionic, cationic, or amphoteric surfactant.

As will be appreciated by those working in the field of pharmaceutical formulation, gels are semisolid, suspension-type systems. Single-phase gels contain organic macromolecules distributed substantially uniformly throughout the carrier liquid, which is typically aqueous, but also, preferably, contain an alcohol and, optionally, an oil. Preferred “organic macromolecules,” i.e., gelling agents, are crosslinked acrylic acid polymers such as the “carbomer” family of polymers, e.g., carboxypolyalkylenes that may be obtained commercially under the Carbopol™ trademark. Also preferred are hydrophilic polymers such as polyethylene oxides, polyoxyethylene-polyoxypropylene copolymers, and polyvinylalcohol; cellulosic polymers such as hydroxypropyl cellulose, hydroxyethyl cellulose, hydroxypropyl methylcellulose, hydroxypropyl methylcellulose phthalate, and methyl cellulose; gums such as tragacanth and xanthan gum; sodium alginate; and gelatin. In order to prepare a uniform gel, dispersing agents such as alcohol or glycerin can be added, or the gelling agent can be dispersed by trituration, mechanical mixing or stirring, or combinations thereof.

Lotions, which are preferred for delivery of cosmetic agents, are preparations to be applied to the skin surface without friction, and are typically liquid or semiliquid preparations in which solid particles, including the active agent, are present in a water or alcohol base. Lotions are usually suspensions of solids, and preferably, for the present purpose, comprise a liquid oily emulsion of the oil-in-water type. Lotions are preferred formulations herein for treating large body areas, because of the ease of applying a more fluid composition. It is generally necessary that the insoluble matter in a lotion be finely divided. Lotions will typically contain suspending agents to produce better dispersions as well as compounds useful for localizing and holding the active agent in contact with the skin, e.g., methylcellulose, sodium carboxymethyl-cellulose, or the like.

Pastes are semisolid dosage forms in which the active agent is suspended in a suitable base. Depending on the nature of the base, pastes are divided between fatty pastes or those made from a single-phase aqueous gels. The base in a fatty paste is generally petrolatum or hydrophilic petrolatum or the like. The pastes made from single-phase aqueous gels generally incorporate carboxymethylcellulose or the like as a base.

Plasters are comprised of a pasty mixture that is spread on the body, either directly or after being saturated into a base material such as cloth. Medications, including the pharmacologically active bases of the invention, may be dissolved or dispersed within the plaster to make a medicated plaster.

Bioadhesives are preparations that adhere to surfaces of body tissues. Polymeric bioadhesive formulations are well known in the art; see, for example, Heller et al., “Biodegradable polymers as drug delivery systems”, in Chasin, M. and Langer, R., eds.: Dekker, New York, pp. 121-161 (1990); and U.S. Pat. No. 6,201,065. Suitable non-polymeric bioadhesives are also known in the art, including certain fatty acid esters (U.S. Pat. No. 6,228,383).

Formulations may also be prepared with liposomes, micelles, and microspheres. Liposomes are microscopic vesicles having a lipid wall comprising a lipid bilayer, and can be used as drug delivery systems herein as well. Generally, liposome formulations are preferred for poorly soluble or insoluble pharmaceutical agents. Liposomal preparations for use in the instant invention include cationic (positively charged), anionic (negatively charged) and neutral preparations. Cationic liposomes are readily available. For example, N[1-2,3-dioleyloxy)propyl]-N,N,N-triethylammonium (DOTMA) liposomes are available under the tradename Lipofectin™ (GIBCO BRL, Grand Island, N.Y.). Similarly, anionic and neutral liposomes are readily available as well, e.g., from Avanti Polar Lipids (Birmingham, Ala.), or can be easily prepared using readily available materials. Such materials include phosphatidyl choline, cholesterol, phosphatidyl ethanolamine, dioleoylphosphatidyl choline (DOPC), dioleoylphosphatidyl glycerol (DOPG), dioleoylphoshatidyl ethanolamine (DOPE), among others. These materials can also be mixed with DOTMA in appropriate ratios. Methods for making liposomes using these materials are well known in the art.

Micelles are known in the art to be comprised of surfactant molecules arranged so that their polar headgroups form an outer spherical shell, while the hydrophobic, hydrocarbon chains are oriented towards the center of the sphere, forming a core. Micelles form in an aqueous solution containing surfactant at a high enough concentration so that micelles naturally result. Surfactants useful for forming micelles include, but are not limited to, potassium laurate, sodium octane sulfonate, sodium decane sulfonate, sodium dodecane sulfonate, sodium lauryl sulfate, docusate sodium, decyltrimethylammonium bromide, dodecyltrimethylammonium bromide, tetradecyltrimethylammonium bromide, tetradecyltrimethylammonium chloride, dodecylammonium chloride, polyoxyl 8 dodecyl ether, polyoxyl 12 dodecyl ether, nonoxynol 10 and nonoxynol 30. Micelle formulations can be used in conjunction with the present invention either by incorporation into the reservoir of a topical or transdermal delivery system, or into a formulation to be applied to the body surface.

Microspheres, similarly, may be incorporated into the present formulations and drug delivery systems. Like liposomes and micelles, microspheres essentially encapsulate a drug or drug-containing formulation. Microspheres are generally, although not necessarily, formed from synthetic or naturally occurring biocompatible polymers, but may also be comprised of charged lipids such as phospholipids. Preparation of microspheres is well known in the art and described in the pertinent texts and literature.

Various additives, known to those skilled in the art, may be included in the topical formulations. For example, solvents, including relatively small amounts of alcohol, may be used to solubilize certain formulation components. Although the pharmacologically active bases herein do penetrate into the skin and have in fact been described as skin permeation enhancers, it may be desirable, particularly with weaker bases, to include an added permeation enhancer in the formulation. Examples of suitable enhancers include, but are not limited to, ethers such as diethylene glycol monoethyl ether (available commercially as Transcutol™) and diethylene glycol monomethyl ether; surfactants such as sodium laureate, sodium lauryl sulfate, cetyltrimethylammonium bromide, benzalkonium chloride, Poloxamer (231, 182, 184), Tween (20, 40, 60, 80), and lecithin (U.S. Pat. No. 4,783,450); alcohols such as ethanol, propanol, octanol, benzyl alcohol, and the like; polyethylene glycol and esters thereof such as polyethylene glycol monolaurate (PEGML; see, e.g., U.S. Pat. No. 4,568,343); amides and other nitrogenous compounds such as urea, dimethylacetamide (DMA), dimethylformamide (DMF), 2-pyrrolidone, 1-methyl-2-pyrrolidone, ethanolamine, diethanolamine and triethanolamine; terpenes; alkanones; and organic acids, particularly citric acid and succinic acid. Azone™ and sulfoxides such as DMSO and C.sub.10 MSO may also be used, but are less preferred.

Most preferred enhancers are those lipophilic co-enhancers typically referred to as “plasticizing” enhancers, i.e., enhancers that have a molecular weight in the range of about 150 to 1000, an aqueous solubility of less than about 1 wt. %, preferably less than about 0.5 wt. %, and most preferably less than about 0.2 wt. %. The Hildebrand solubility parameter a of plasticizing enhancers is in the range of about 2.5 to about 10, preferably in the range of about 5 to about 10. Such enhancers are described in detail in co-pending, commonly assigned U.S. patent application Ser. No. 09/738,410, filed on Dec. 14, 2000, and in International Patent Application No. PCT/US00/34483, published Jun. 21, 2001 as WO 01/43775 A2. Preferred lipophilic enhancers are fatty esters, fatty alcohols, and fatty ethers. Examples of specific and most preferred fatty acid esters include methyl laurate, ethyl oleate, propylene glycol monolaurate, propylene glycerol dilaurate, glycerol monolaurate, glycerol monooleate, isopropyl n-decanoate, and octyldodecyl myristate. Fatty alcohols include, for example, stearyl alcohol and oleyl alcohol, while fatty ethers include compounds wherein a diol or triol, preferably a C.sub.2-C.sub.4 alkane diol or triol, are substituted with one or two fatty ether substituents.

Additional permeation enhancers will be known to those of ordinary skill in the art of topical drug delivery, and/or are described in the pertinent texts and literature. See, e.g., Percutaneous Penetration Enhancers, Smith et al., eds. (CRC Press, 1995).

The present formulations may also include conventional additives such as opacifiers, antioxidants, fragrance, colorants, gelling agents, thickening agents, stabilizers, surfactants, and the like. Other agents may also be added, such as antimicrobial agents, to prevent spoilage upon storage, i.e., to inhibit growth of microbes such as yeasts and molds. Suitable antimicrobial agents are typically selected from the group consisting of the methyl and propyl esters of p-hydroxybenzoic acid (i.e., methyl and propyl paraben), sodium benzoate, sorbic acid, imidurea, and combinations thereof.

The formulations may also contain irritation-mitigating additives to minimize or eliminate the possibility of skin irritation or skin damage resulting from the pharmacologically active base or other components of the composition. Suitable irritation-mitigating additives include, for example: .alpha.-tocopherol; monoamine oxidase inhibitors, particularly phenyl alcohols such as 2-phenyl-1-ethanol; glycerin; salicylic acids and salicylates; ascorbic acids and ascorbates; ionophores such as monensin; amphiphilic amines; ammonium chloride; N-acetylcysteine; cis-urocanic acid; capsaicin; and chloroquine. The irritant-mitigating additive, if present, may be incorporated into the present formulations at a concentration effective to mitigate irritation or skin damage, typically representing not more than about 20 wt. %, more typically not more than about 5 wt. %, of the composition.

The pharmacologically active base may also be administered through the skin or mucosal tissue using a conventional skin patch, wherein the agent is contained within a laminated structure that serves as a drug delivery device to be affixed to the body surface. In such a structure, the pharmaceutical formulation is contained in a layer, or “reservoir,” underlying an upper backing layer. The laminated structure may contain a single reservoir, or it may contain multiple reservoirs.

In one embodiment, the reservoir comprises a polymeric matrix of a pharmaceutically acceptable adhesive material that serves to affix the system to the skin during drug delivery; typically, the adhesive material is a pressure-sensitive adhesive (PSA) that is suitable for long-term skin contact, and that should be physically and chemically compatible with the pharmacologically active base and any carriers, vehicles or other additives that are present. Examples of suitable adhesive materials include, but are not limited to, the following: polyethylenes; polysiloxanes; polyisobutylenes; polyacrylates; polyacrylamides; polyurethanes; plasticized ethylene-vinyl acetate copolymers; and tacky rubbers such as polyisobutene, polybutadiene, polystyrene-isoprene copolymers, polystyrene-butadiene copolymers, and neoprene (polychloroprene). Preferred adhesives are polyisobutylenes.

The backing layer functions as the primary structural element of the transdermal system and provides the device with flexibility and, preferably, occlusivity. The material used for the backing layer should be inert and incapable of absorbing drug, base, or components of the formulation contained within the device. The backing is preferably comprised of a flexible elastomeric material that serves as a protective covering to prevent loss of drug and/or vehicle via transmission through the upper surface of the patch, and will preferably impart a degree of occlusivity to the system, such that the area of the body surface covered by the patch becomes hydrated during use. The material used for the backing layer should permit the device to follow the contours of the skin and be worn comfortably on areas of skin such as at joints or other points of flexure, that are normally subjected to mechanical strain, with little or no likelihood of the device disengaging from the skin due to differences in the flexibility or resiliency of the skin and the device. The materials used as the backing layer are either occlusive or permeable, as noted above, although occlusive backings are preferred, and are generally derived from synthetic polymers (e.g., polyester, polyethylene, polypropylene, polyurethane, polyvinylidine chloride, and polyether amide), natural polymers (e.g., cellulosic materials), or macroporous woven and nonwoven materials.

During storage and prior to use, the laminated structure includes a release liner. immediately prior to use, this layer is removed from the device so that the system may be affixed to the skin. The release liner should be made from a drug/vehicle impermeable material, and is a disposable element that serves only to protect the device prior to application. Typically, the release liner is formed from a material impermeable to the pharmacologically active agent and the base, and which is easily stripped from the patch prior to use.

In an alternative embodiment, the active agent-containing reservoir and skin contact adhesive are present as separate and distinct layers, with the adhesive underlying the reservoir. In such a case, the reservoir may be a polymeric matrix as described above. Alternatively, the reservoir may be comprised of a liquid or semisolid formulation contained in a closed compartment or “pouch,” or it may be a hydrogel reservoir, or may take some other form. Hydrogel reservoirs are particularly preferred herein. As will be appreciated by those skilled in the art, hydrogels are macromolecular networks that absorb water and thus swell but do not dissolve in water. That is, hydrogels contain hydrophilic functional groups that provide for water absorption, but the hydrogels are comprised of crosslinked polymers that give rise to aqueous insolubility. Generally, then, hydrogels are comprised of crosslinked hydrophilic polymers such as a polyurethane, a polyvinyl alcohol, a polyacrylic acid, a polyoxyethylene, a polyvinylpyrrolidone, a poly(hydroxyethyl methacrylate) (poly(HEMA)), or a copolymer or mixture thereof. Particularly preferred hydrophilic polymers are copolymers of HEMA and polyvinylpyrrolidone.

Additional layers, e.g., intermediate fabric layers and/or rate-controlling membranes, may also be present in any of these drug delivery systems. Fabric layers may be used to facilitate fabrication of the device, while a rate-controlling membrane may be used to control the rate at which a component permeates out of the device. The component may be an active agent, an enhancer, or some other component contained in the drug delivery system. A rate-controlling membrane, if present, will be included in the system on the skin side of one or more of the drug reservoirs. The materials used to form such a membrane are selected to limit the flux of one or more components contained in the drug formulation. Representative materials useful for forming rate-controlling membranes include polyolefins such as polyethylene and polypropylene, polyamides, polyesters, ethylene-ethacrylate copolymer, ethylene-vinyl acetate copolymer, ethylene-vinyl methylacetate copolymer, ethylene-vinyl ethylacetate copolymer, ethylene-vinyl propylacetate copolymer, polyisoprene, polyacrylonitrile, ethylene-propylene copolymer, and the like.

Generally, the underlying surface of the transdermal device, i.e., the skin contact area, has an area in the range of about 0.25 cm² to 200 cm², preferably 1 cm² to 25 cm², more preferably 2 cm² to 10 cm². That area will vary, of course, with the size of the area to be treated. Larger patches will be necessary to accommodate larger afflicted areas, whereas smaller patches can be used for smaller afflicted areas.

Such drug delivery systems may be fabricated using conventional coating and laminating techniques known in the art. For example, adhesive matrix systems can be prepared by casting a fluid admixture of adhesive, active agent and vehicle onto the backing layer, followed by lamination of the release liner. Similarly, the adhesive mixture may be cast onto the release liner, followed by lamination of the backing layer. Alternatively, the drug reservoir may be prepared in the absence of drug or excipient, and then loaded by “soaking” in a drug/vehicle mixture. In general, these patches are fabricated by solvent evaporation, film casting, melt extrusion, thin film lamination, die cutting, or the like. The active agent will generally be incorporated into the device during patch manufacture rather than subsequent to preparation of the device.

In a preferred delivery system, an adhesive overlayer that also serves as a backing for the delivery system is used to better secure the patch to the body surface. This overlayer is sized such that it extends beyond the drug reservoir so that adhesive on the overlayer comes into contact with the body surface. The overlayer is useful because the adhesive/drug reservoir layer may lose its adhesion a few hours after application due to hydration. By incorporating such adhesive overlayer, the delivery system remains in place for the required period of time.

Other types and configurations of topically applied drug delivery systems may also be used in conjunction with the present invention, as will be appreciated by those skilled in the art of topical drug delivery. See, for example, Ghosh, Transdermal and Topical Drug Delivery Systems (Interpharm Press, 1997), particularly Chapters 2 and 8.

V. Administration:

The method of delivery of the active agent may vary, but necessarily involves application of a formulation of the invention to an area of body surface affected with one or more inflamed area. A cream, ointment, paste, plaster, or lotion may be spread on the afflicted area and gently rubbed in. Similarly, a polymeric or other bioadhesive formulation may be spread or dabbed on the afflicted area. A solution may be applied in the same ways, but more typically will be applied with a dropper, swab, or the like, and carefully applied to the afflicted area. Petrolatum may be spread on the skin surrounding the afflicted area to protect it from possible irritation during treatment.

The dose regimen will depend on a number of factors that may readily be determined, such as the size of the afflicted area and the responsiveness of the afflicted area to treatment, but will normally be one or more doses per day, with a course of treatment lasting from several days to several months, or until a cure is effected or a significant diminution in the size of the afflicted area is achieved. One of ordinary skill may readily determine optimum dosages, dosing methodologies and repetition rates. In general, it is contemplated that the formulation will be applied one to four times daily. With a skin patch, which is a preferred embodiment, the device is generally maintained in place on the body surface throughout a drug delivery period, typically in the range of 8 to 72 hours, and replaced as necessary.

It is to be understood that while the invention has been described in conjunction with the preferred specific embodiments thereof, the foregoing description is intended to illustrate and not limit the scope of the invention. Other aspects, advantages, and modifications will be apparent to those skilled in the art to which the invention pertains. Furthermore, the practice of the present invention will employ, unless otherwise indicated, conventional techniques of drug formulation, particularly topical and transdermal drug formulation, which are within the skill of the art. Such techniques are fully explained in the literature. See Remington: The Science and Practice of Pharmacy, cited supra, as well as Goodman & Gilman's The Pharmacological Basis of Therapeutics, 9th Ed. (New York: McGraw-Hill, 1996).

All patents, patent applications, and publications mentioned herein are hereby incorporated by reference in their entireties.

Experimental

The practice of the present invention will employ, unless otherwise indicated, conventional techniques of pharmaceutical formulation and the like, which are within the skill of the art. Such techniques are fully explained in the literature. In the following examples, efforts have been made to ensure accuracy with respect to numbers used (e.g., amounts, temperatures, etc.) but some experimental error and deviation should be accounted for. Unless otherwise indicated, temperature is in degrees Celsius and pressure is at or near atmospheric pressure at sea level. All reagents were obtained commercially unless otherwise indicated.

### EXAMPLE 1

A topical gel of the invention is prepared by conventional pharmaceutical methods. The indicated amounts of the following ingredients are used:

| Ingredient | Amount | |
|---|---|---|
| Purified water | 600 | grams |
| Polyethylene glycol | 400 | grams |
| Potassium hydroxide | 0.01 | gram |
| Edetate disodium | 0.1 | gram |
| Carbomer 934P | 12.5 | grams |
| Poloxamer 407 | 2.0 | grams |
| Polysorbate 40 | 2.0 | grams |
| Butylated hydroxytoluene | 0.5 | grams |
| Benzyl alcohol | 10.0 | grams |
| Isomalt | 100.0 | grams |

The Carbomer 934P and the edetate disodium are added to 250 ml of the purified water, and the mixture is homogenized at low speed until the Carbomer is dispersed.

Next, the polaxamer 407, mixed with 250 ml of the purified water, is added to the Carbomer mixture, and the resulting mixture is homogenized at low speed. The potassium hydroxide, dissolved in 100 ml of purified water, is added to this mixture, and the resulting mixture (Mixture 1) is homogenized at low speed. In a separate container, the polysorbate 40 and the butylated hydroxytoluene are added to the polyethylene glycol, and the resulting mixture is heated to 65. degree. C. and maintained at this temperature until all the compounds are dissolved; this mixture is then allowed to cool to room temperature, at which time the benzyl alcohol is added, and the resulting mixture is homogenized at low speed. This mixture is then added to Mixture 1, and the resulting mixture is mixed at low speed until it is homogeneous, forming a gel of the invention.

### EXAMPLE 2

A topical cream of the invention is prepared by conventional pharmaceutical methods. The indicated amounts of the following ingredients are used:

| Ingredient | Amount | |
|---|---|---|
| Purified water | 370 | grams |
| White petrolatum | 250 | grams |
| Stearyl alcohol | 250 | grams |
| Propylene glycol | 120 | grams |
| Sodium lauryl sulfate | 10 | grams |
| Methylparaben | 0.25 | gram |
| Propylparaben | 0.15 | gram |
| Potassium hydroxide | 0.01 | gram |
| Isomaltitol | 50.0 | grams |

The stearyl alcohol and the white petrolatum are melted together on a steam bath, and then maintained at a temperature of approximately 75° C. The other ingredients are then added, after previously having been dissolved in the purified water and warmed to 75° C., and the resulting mixture is stirred until it congeals into a cream of the invention.

### EXAMPLE 3

A skin patch of the invention may be prepared by conventional pharmaceutical methods. A square piece of sterile, finely woven gauze one centimeter on each side is placed in the center of a square piece of occlusive surgical adhesive tape two centimeters on each side. To the gauze is applied 0.4 ml of the gel of Example 1; the gel is allowed to soak into the gauze. This skin patch of the invention is used within three hours of preparation.

### EXAMPLE 4

A topical composition including a mixture of isomaltitol, an hydrophilic ointment base, sodium acid phosphate moisturizing agent, a witch hazel extract carrier, glycerine, apricot kernel oil and DL-panthenol, as the pharmaceutically acceptable carrier and vitamins A and D3, ascorbyl palmitate, and vitamin E acetate as the ingredients which have antioxidant properties and/or regulate cell differentiation and/or cell proliferation was prepared by cold compounding.

The topical composition was prepared by first placing the hydrophilic ointment base in a stainless steel bowl and mixing briskly until the ointment became creamy. Then, the sodium acid phosphate, panthenol, ascorbyl palmitate, glycerine, apricot kernel oil, quercetin, witch hazel extract, and vitamin E acetate were added, in that order. After each ingredient was added, mixing was continued until all traces of dry ingredients disappeared and a substantially homogeneous mixture was obtained. The final colour was a consistent yellow and the cream had the consistency of cake frosting. The mixture was then placed in a sterile container. All containers and tools which contacted the topical composition or its ingredients were sterilized with, for example, a benzalkonium chloride, a sodium hypochlorite solution, or an iodine-based disinfectant.

This composition may be topically administered by applying a thin film of the composition to the areas of the skin afflicted with reactive and inflammatory dermatoses such as eczema and/or psoriasis. The topical composition may be applied three times daily, e. g., in the morning, at noon and before retiring. Patients applying the topical composition should experience less severe burning, irritation and redness in the areas of skin that were treated.

A standard reference text on pharmaceutical formulations, Remington's Pharmaceutical Sciences, 15th Ed. , Mack Publishing Co. 1990, is also incorporated herein by reference in its entirety.

Although the present invention has been described and exemplified in terms of certain preferred embodiments, other embodiments will be apparent to those skilled in the art. The invention is, therefore, not limited to the particular embodiments described and exemplified, but is capable of modification or variation without departing from the spirit of the invention, the full scope of which is delineated by the appended claims.

The active compounds of the invention are administered in an amount of 1 to 100 mg per kilogram body weight depending on the condition of the patient, route of administration, age and body weight of the patient, and other considerations made by the physician. The most important aspect hereby is the serum concentration which may be 0.1 to 100 mM of active compounds, in accordance with the present findings.

### REFERENCES

Fullerton A, Stucker M, Wilhelm K P, Wardell K et al. Guidelines for visualization of cutaneous blood flow by laser Doppler perfusion imaging. A report from the Standardization Group of the European Society of Contact Dermatitis based upon the HIRELADO European community project. Contact Dermatitis. 2002 March; 46(3):129-40.

Ilias M A. Single exposure phototesting and assessment of pigmented skin lesions. Dissertation No. 839, Linkoping studies in Science and Technology. 2003.

Pinnagoda J, Tupker R A, Agner T, Serup J. Guidelines for transepidermal water loss (TEWL) measurement. A report from the Standardization Group of the European Society of Contact Dermatitis. Contact Dermatitis. 1990 March; 22(3):164-78.

Serup J, Jemec GBE. Handbook of non-invasive methods and the skin. CRC Press Inc., Boca Raton, Fla., USA. 1995.

Sjögren F, Groth O, Anderson C. Acetone has anti-inflammatory effects on experimental contact reactions. Contact Dermatitis. 1999 July; 41(1):22-9.

York M, Griffiths H A, Whittle E, Basketter D A. Evaluation of a human patch test for the identification and classification of skin irritation potential. Contact dermatitis. 1996; 34:204-212.

1. The use of one or more of the compounds of the group consisting of 4-thiouridine, isomaltitol, and uridine in the preparation of topical, therapeutically effective compositions for treating psoriasis, atopic and contact dermititis, eczema, seborrhoea, stasis dermatitis and stasis ulcers, as well as allergic rhinitis, symptoms of irritation, dryness, itching, erythema, swelling, due to occupational exposure to irritants or as a result of exposure to UV light including sunshine and artificial lamps, or as a result of various cosmetological procedures including so called peeling, with the exception of the use of uridine in the treatment of inflammatory conditions caused by bacterial infection.

2. The use according to claim 1, wherein the therapeutically effective compound is 4-thiouridine.

3. The use according to claim 1, wherein the therapeutically effective compound is isomaltitol.

4. The use according to claim 1, wherein the therapeutically effective compound is uridine.

5. The use according to claim 1, wherein the therapeutically effective dose is 1 to 100 mg per kg bodyweight.

6. The use according to claim 1, wherein isomalt consisting of isomaltitol (6-O-α-D-glucopyranosyl-D-sorbitol, GPS) and glucomannitol (6-O-α-D-glucopyranosyl-D-mannitol, GPM) is used as an active ingredient.

7. A method for treatment of psoriasis, atopic and contact dermatitis, eczema, seborrhea, stasis dermatitis and stasis ulcers, as well as allergic rhinitis, wherein the therapeutically effective amount of one or more of the compounds of the group consisting of 4-thiouridine, isomaltitol, and uridine is administered, with the exception of the use of uridine in the treatment of inflammatory conditions caused by bacterial infection.

8. A method according to claim 7, wherein the active compound is administered in such an amount that the serum concentration thereof is 0.1 to 100 mM.

9. A method according to claim 8, wherein isomaltitol is present in the form of isomalt.

10. Compositions in accordance with claim 1, for topical administration for treating all and varied symptoms of psoriasis, atopic and contact dermatitis, eczema, seborrhea, stasis dermatitis and stasis ulcers, as well as allergic rhinitis, dyshidrotic eczema, nummular eczema, lichen simplex chronicus, asteatotic eczema, eczema caused by irritants, and seborrheic dermatitis. Hand eczema can also be related to occupational exposure to various irritants and comprising one or more of the compounds of the group consisting of 4-thiouridine, isomaltitol, and uridine,

11. A composition according to claim 10, wherein isomaltitol is present in the form of isomalt.
